(19) United States
(12) Patent Application Publication
(10) Pub. No.: US 2005/0008323 A1
(43) Pub. Date: Jan. 13, 2005

(54) Apparatus and method of signal reproduction using a digital visual interface/high definition multimedia interface compatible connector

(76) Inventor: Dong-hoon Han, Suwon-si (KR)

Correspondence Address: STANZIONE & KIM, LLP, 1740 N STREET, N.W., FIRST FLOOR, WASHINGTON, DC, 20036, US

(21) Appl. No.: 10/851,125

(22) Filed: May 24, 2004

(30) Foreign Application Priority Data
May 26, 2003 (KR) 2003-33345

(51) Int. Cl. A61N 1/18

(52) U.S. Cl. 386/37

(57) ABSTRACT

An apparatus and method of signal reproduction with a DVI/HDMI compatible connector. The apparatus includes an HDMI audio detector that determines whether an HDMI audio signal is included in a signal input via the DVI/HDMI compatible connector, a switch that selects one of an HDMI audio signal input via the DVI/HDMI compatible connector and a DVI audio signal input via a separate DVI audio connector, according to the result detected by the audio detector, and an audio decoder that decodes the audio signal selected by the switch.

### CROSS-REFERENCE TO RELATED APPLICATIONS

This application claims the priority of Korean Patent Application No. 2003-33345, filed on May 26, 2003, in the Korean Intellectual Property Office, the disclosure of which is incorporated herein in its entirety by reference.

### BACKGROUND OF THE INVENTION

1. Field of the Invention

The present general inventive concept relates to digital signal processing, and more particularly, to an apparatus and method of signal reproduction using a digital visual interface (DVI)/high definition multimedia interface (HDMI) compatible connector, in which an input signal is automatically detected and reproduced.

2. Description of the Related Art

DVI and HDMI are specifications developed as graphic interfaces and employed by digital video display devices in general.

The DVI specification, published by the digital display working group (DDWG), supports only digital video sources, from which digital video signals are input to a digital video display device via the DVI.

The HDMI specification, founded by a consortium of several connector-chip manufacturers, was designed as an enhancement of the DVI specification to lead to a real entertainment era.

While a DVI relays, from a receiver, uncompressed digital video signals to digital display devices, an HDMI offers an interface supporting not just uncompressed high-definition videos, but also multi-channel audio at a high speed of up to 5 Gbps. In addition, the HDMI connector (or chip) has been developed to be compatible with the DVI, so it is also referred to as a DVI/HDMI compatible connector, by which a DVI video signal is also relayed from a corresponding signal source to a display device.

FIG. 1 is a schematic block diagram of a conventional signal processing apparatus with a DVI/HDMI compatible connector. The DVI/HDMI compatible connector 100 allows DVI signals and HDMI signals to be input to the apparatus. A signal input by the compatible DVI/HDMI connector 100 is forwarded to a decoder 110 that decodes HDMI and DVI video signals. However, if a DVI signal is generated by a corresponding source along with an audio signal, the audio signal is not included in the DVI signal and needs its own dedicated connector 120 to be relayed to the apparatus. Accordingly, a user has to determine whether the input signal is the DVI signal or the HDMI signal, and then turn a switch 130 to select the corresponding audio signal, a DVI audio signal or an HDMI audio signal, according to the result. The audio signal selected by the switch 130 is sent to an audio decoder 140 to be decoded and output.

However, the conventional apparatus for signal reproduction with the compatible DVI/HDMI connector 100, as shown In FIG. 1, forces a user to determine whether the input signal is the DVI signal or the HDMI signal and then to correspondingly make the proper settings for the apparatus to process the determined signal.

### SUMMARY OF THE INVENTION

Accordingly, it is an aspect of the present general inventive concept to provide an apparatus and method of signal reproduction using a DVI/HDMI compatible connector, which automatically detects a type of an input signal.

Additional aspects and advantages of the present general inventive concept will be set forth in part in the description which follows and, in part, will be obvious from the description, or may be learned by practice of the general inventive concept.

The foregoing and/or other aspects of the general inventive concept are achieved by providing an apparatus that can provide signal reproduction with a digital visual interface (DVI)/high definition multimedia interface (HDMI) compatible connector, the apparatus includes an HDMI audio detector that can determine whether an HDMI audio signal is included in a signal input via the DVI/HDMI compatible connector, a switch that can select one of an HDMI audio signal input via the DVI/HDMI compatible connector and a DVI audio signal input via a separate DVI audio connector, according to the result detected by the audio detector, and an audio decoder that can decode the audio signal selected by the switch.

It is an aspect of the general inventive concept that the HDMI audio detector can determine whether the HDMI audio signal is included in the input signal by reading information from an audio header of a data frame of the input signal.

It is another aspect of the general inventive concept that the switch can be turned to select the HDMI audio signal input via the DVI/HDMI compatible connector, if the audio detector has detected the audio signal from the input signal, and otherwise, turned to select the DVI audio signal output by the DVI audio connector.

It is another aspect of the general inventive concept that the apparatus may further include a MICOM that can provide a selection signal to the switch, according to the result detected by the audio detector.

The foregoing and/or other aspects of the present general inventive concept may also be achieved by providing an apparatus that can provide signal reproduction, the apparatus including a digital visual interface (DVI)/high definition multimedia interface (HDMI) compatible connector that can allow a DVI video signal or a HDMI signal to be input, a DVI audio connector that can allow a DVI audio signal to be input, a video decoder that can decode the DVI video signal or a video signal included in the HDMI signal input by the DVI/HDMI compatible connector, an HDMI audio detector that can detect an HDMI audio signal from the signal output by the DVI/HDMI compatible connector, a switch that can select one of the HDMI audio and the DVI audio signals, according to the result detected by the HDMI audio detector, and an audio decoder that can decode the audio signal selected by the switch.

It is another aspect of the general inventive concept that the apparatus can further include a MICOM that can control the switch to select the HDMI audio signal input via the DVI/HDMI compatible connector if the HDMI audio detector has detected the audio signal from the signal input by the DVI/HDMI compatible connector, and, otherwise, to select the DVI audio signal input via the DVI connector.

It is another aspect of the general inventive concept that the HDMI audio detector can determine whether the signal input via the DVI/HDMI compatible connector includes an audio signal by reading information from an audio header of a data frame of the input signal.

The foregoing and/or other aspects of the present general inventive concept may also be achieved by providing a method of reproducing audio signals input by a digital visual interface (DVI)/high definition multimedia interface (HDMI) compatible connector, the method may include detecting whether a signal input via the DVI/HDMI compatible connector is an HDMI signal or a DVI signal, and decoding an HDMI audio signal input via the DVI/HDMI compatible connector, if the HDMI signal has been detected, and, otherwise, decoding an audio signal input via a separate DVI audio connector.

It is an aspect of the general inventive concept that detecting whether the signal input by the DVI/HDMI compatible connector is the HDMI signal or the DVI signal, can include reading information from an audio header of a data frame of the detected signal, and determining that the HDMI signal has been detected, if the information indicates that an audio data is included in the data frame, and, otherwise, determining that the DVI signal has been detected.

### BRIEF DESCRIPTION OF THE DRAWINGS

These and/or other aspects and advantages of the present general inventive concept will become apparent and more readily appreciated from the following description of the embodiments, taken in conjunction with the accompanying drawings of which:

FIG. 1 is a schematic block diagram of a conventional apparatus for signal reproduction with a DVI/HDMI compatible connector;

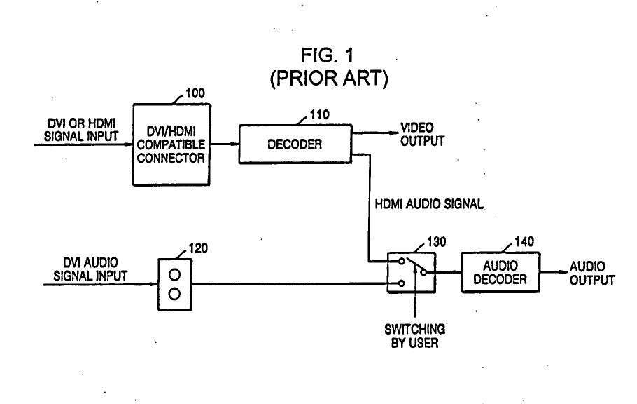

FIG. 2 is a schematic block diagram of an apparatus to provide signal reproduction with a DVI/HDMI compatible connector, according to an embodiment of the present general inventive concept; and

FIG. 3B is a flowchart of a method of reproducing a DVI/HDMI audio signal, according to another embodiment of the present general inventive concept.

### DETAILED DESCRIPTION OF THE PREFERRED EMBODIMENTS

Reference will now be made in detail to the embodiments of the present general inventive concept, examples of which are illustrated in the accompanying drawings, wherein like reference numerals refer to the like elements throughout. The embodiments are described below to explain the present general inventive concept by referring to the figures.

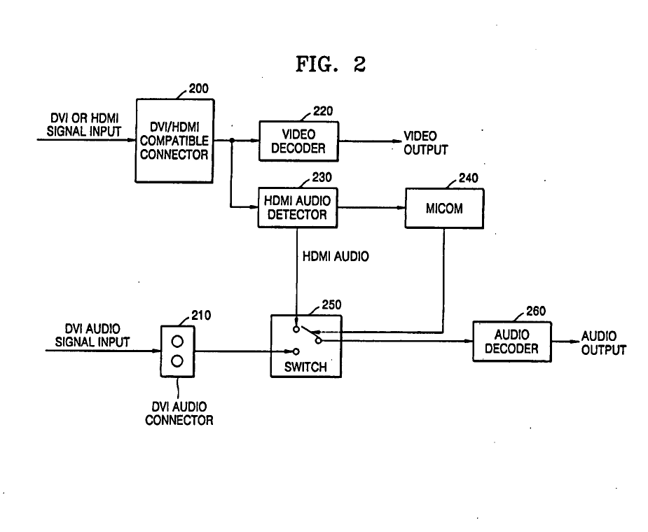

FIG. 2 is a block diagram of an apparatus to provide signal reproduction with a DVI/HDMI compatible connector. The apparatus can include a DVI/HDMI compatible connector 200, a DVI audio connector 210, a video decoder 220, an HDMI audio detector 230, a micom 240, a switch 250, and an audio decoder 260.

The DVI/HDMI compatible connector 200 can allow a DVI signal, as well as an HDMI signal, to be input from an exterior signal source (not shown) to the apparatus, the DVI signal and the HDMI signal being respectively formatted according to the DVI and HDMI specifications.

The DVI audio connector 210 can be separated from the DVI/HDMI compatible connector 200, allowing a DVI audio signal to be input.

The video decoder 220 can decode a video signal relayed by the DVI/HDMI compatible connector 200.

The HDMI audio detector 230 can detect whether an audio signal (or an HDMI audio signal) is included in the signal output by the DVI/HDMI compatible connector 200. To do this, the HDMI audio detector 230 can read information from an audio header of a data frame of the signal output by the DVI/HDMI compatible connector 200 and can determine from the information whether an audio signal is included in the signal.

The MICOM 240 can generate a selection signal to select one of the DVI audio signal output by the DVI audio connector 210 and the HDMI audio signal detected by the HDMI audio detector 230, according to the result detected by the audio detector 230. If the HDMI audio signal has been detected by the HDMI audio detector 230, the MICOM 240 can determine that the signal input by the DVI/HDMI compatible connector 200 is the HDMI signal and can generate a HDMI selection signal. On the other hand, if an audio signal has not been detected by the HDMI audio detector 230, the MICOM 240 can determine that the signal input by the HDMI compatible connector 200 is the DVI signal and can generate a DVI selection signal. In this embodiment of FIG. 2, though the MICOM 240 is implemented as a separate component in the apparatus, it may also be incorporated with the audio detector 230.

The switch 250 can be turned to connect the DVI or HDMI audio signal output by the DVI audio connector 210 or detected by the HDMI audio detector 230 respectively, according to the (DVI or HDMI) selection signal generated by the MICOM 240.

The audio decoder 260 can decode the audio signal selected by the switch 250 and can output the result.

In the apparatus described above, the HDMI audio detector 230 and the video decoder 220 may be incorporated with each other into a single HDMI decoder.

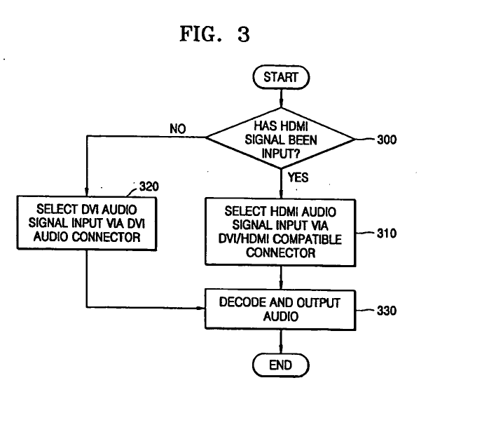

FIG. 3 is a flowchart of a method of reproducing audio signals, which can be performed by the apparatus of FIG. 2.

First, the MICOM 240 can determine whether a signal input by the DVI/HDMI compatible connector 200 is the HDMI signal in operation 300. The MICOM 240 can read information from an audio header of a data frame of the signal and can determine from the information whether the signal is a DVI signal or an HDMI signal. That is, if the information indicates that the input signal includes an audio signal, the MICOM 240 can determine that the input signal is the HDMI signal, and, otherwise, the input signal is determined to be the DVI signal.

When the HDMI signal is detected in operation 300, the MICOM 240 can extract an (HDMI) audio signal from the signal input by the DVI/HDMI compatible connector 200 in operation 310.

When the DVI signal is detected in operation 300, the MICOM 240 can allow the DVI audio signal to be accepted through the DVI audio connector 210 in operation 320.

In operation 330, the audio decoder 260 can decode the audio signal, which was extracted in operation 310 or accepted in operation 320, and can output the result.

As described above, the apparatus to provide signal reproduction with the DVI/HDMI compatible connector can automatically detect the source (or the type) of the input signal and properly process the corresponding signal.

Although a few embodiments of the present general inventive concept have been shown and described, it will be appreciated by those skilled in the art that changes may be made in these embodiments without departing from the principles and spirit of the general inventive concept, the scope of which is defined in the appended claims and their equivalents.

1. An apparatus to provide signal reproduction with a digital visual interface (DVI)/high definition multimedia interface (HDMI) compatible connector, the apparatus comprising:
an HDMI audio detector that determines whether an HDMI audio signal is included in a signal input via the DVI/HDMI compatible connector;
a switch that selects one of an HDMI audio signal input via the DVI/HDMI compatible connector and a DVI audio signal input via a separate DVI audio connector, according to the result detected by the audio detector; and
an audio decoder that decodes the audio signal selected by the switch.

2. The apparatus of claim 1, wherein the HDMI audio detector determines whether the HDMI audio signal is included in the input signal by reading information from an audio header of a data frame of the input signal.

3. The apparatus of claim 1, wherein the switch is turned to select the HDMI audio signal input via the DVI/HDMI compatible connector, if the audio detector has detected the audio signal from the input signal, and otherwise, the switch is turned to select the DVI audio signal output by the DVI audio connector.

4. The apparatus of claim 3, further comprising:
a MICOM that provides a selection signal to the switch, according to the result detected by the audio detector.

5. The apparatus of claim 4, wherein the MICOM is incorporated within the audio detector as one unit.

6. An apparatus to provide signal reproduction, comprising:
a digital visual interface (DVI)/high definition multimedia interface (HDMI) compatible connector that allows a DVI video signal or a HDMI signal to be input;
a DVI audio connector that allows a DVI audio signal to be input;
a video decoder that decodes the DVI video signal or a video signal included in the HDMI signal input by the DVI/HDMI compatible connector;
an HDMI audio detector that detects an HDMI audio signal from the signal output by the DVI/HDMI compatible connector;
a switch that selects one of the HDMI audio and the DVI audio signals, according to the result detected by the HDMI audio detector; and
an audio decoder that decodes the audio signal selected by the switch.

7. The apparatus of claim 6, further comprising:
a MICOM that controls the switch to select the HDMI audio signal input via the DVI/HDMI compatible connector, if the HDMI audio detector has detected the audio signal from the signal input by the DVI/HDMI compatible connector, and, otherwise, to select the DVI audio signal input via the DVI connector.

8. The apparatus of claim 6, wherein the HDMI audio detector determines whether the signal input via the DVI/HDMI compatible connector includes an audio signal by reading information from an audio header of a data frame of the input signal.

9. The apparatus of claim 6, wherein the HDMI audio detector and the video decoder are incorporated with each other as a single HDMI decoder.

10. An apparatus to provide signal reproduction, comprising:
a digital visual interface/high definition multimedia interface (DVI/HDMI) compatible connector that allows a DVI video signal or a HDMI signal to be input;
a DVI audio connector that allows a DVI audio signal to be input; and
a switch that automatically selects one of the HDMI signal input via the DVI/HDMI compatible connector and the DVI audio signal input via a separate DVI audio connector, according to whether an HDMI signal from the signal output by the DVI/HDMI compatible connector includes an audio signal.

11. A method of reproducing audio signals input by a digital visual interface (DVI)/high definition multimedia interface (HDMI) compatible connector, the method comprising:
detecting whether a signal input via the DVI/HDMI compatible connector is an HDMI signal or a DVI signal; and
decoding an HDMI audio signal input via the DVI/HDMI compatible connector, if the HDMI signal has been detected, and, otherwise, decoding an audio signal input via a separate DVI audio connector.

12. The method of claim 11, wherein detecting whether the signal input by the DVI/HDMI compatible connector is the HDMI signal or the DVI signal, comprises:
reading information from an audio header of a data frame of the detected signal; and
determining that the HDMI signal has been detected, if the information indicates that audio data is included in the data frame, and, otherwise, determining that the DVI signal has been detected.

13. A method of reproducing audio signals, comprising:
determining whether an HDMI audio signal is included in a signal input to DVI/HDMI
compatible connector;
selecting one of an HDMI audio signal input via the DVI/HDMI compatible connector and a DVI audio signal input via a separate DVI audio connector, according to the determining operation; and
decoding the audio signal selected.

14. A computer readable medium performing a method of reproducing audio signals input by a digital visual interface (DVI)/high definition multimedia interface (HDMI) compatible connector, the method comprising:
detecting whether a signal input via the DVI/HDMI compatible connector is an HDMI signal or a DVI signal; and
decoding an HDMI audio signal input via the DVI/HDMI compatible connector, if the HDMI signal has been detected, and, otherwise, decoding an audio signal input via a separate DVI audio connector.

15. A computer readable medium performing a method of reproducing audio signals, the method comprising:
determining whether an HDMI audio signal is included in a signal input to DVI/HDMI
compatible connector;
selecting one of an HDMI audio signal input via the DVI/HDMI compatible connector and a DVI audio signal input via a separate DVI audio connector, according to the determining operation; and
decoding the audio signal selected.
